SEQUENCE LISTING (3 sequences)

SEQ ID NO: 1 - DNA, 20 bp, Artificial sequence
  Chemically synthesized sequence
agagtttgatcmtggctcag

SEQ ID NO: 2 - DNA, 19 bp, Artificial sequence
  Chemically synthesized sequence
ggytaccttgttacgactt

SEQ ID NO: 3 - DNA, 975 bp, Lactobacillus plantarum 16S rDNA
  Lactobacillus plantarum 16S rDNA gene sequence
gtcgaacgaactctggtattgattggtgcttgcatcatgatttacatttgagtgagtggcgaactggtgagtaacacgtgggaaacctgcccagaagcgggggataacacctggaaacagatgctaataccgcataacaacttggaccgcatggtccgagtttgaaagatggcttcggctatcacttttggatggtcccgcggcgtattagctagatggtggggtaacggctcaccatggcaatgatacgtagccgacctgagagggtaatcggccacattgggactgagacacggcccaaactcctacgggaggcagcagtagggaatcttccacaatggacgaaagtctgatggagcaacgccgcgtgagtgaagaagggtttcggctcgtaaaactctgttgttaaagaagaacatatctgagagtaactgttcaggtattgacggtatttaaccagaaagccacggctaactacgtgccagcagccgcggtaatacgtaggtggcaagcgttgtccggatttattgggcgtaaagcgagcgcaggcggttttttaagtctgatgtgaaagccttcggctcaaccgaagaagtgcatcggaaactgggaaacttgagtgcagaagaggacagtggaactccatgtgtagcggtgaaatgcgtagatatatggaagaacaccagtggcgaaggcggctgtctggtctgtaactgacgctgaggctcgaaagtatgggtagcaaacaggattagataccctggtagtccataccgtaaacgatgaatgctaagtgttggagggtttccgcccttcagtgctgcagctaacgcattaagcattccgcctggggagtacggccgcaaggctgaaactcaaaggaattgacgggggcccgcacaagcggtggagcatgtggtttaattcgaagctacgcgaagaaccttaccaggtcttgacatactatgcaaatct

(19) United States
(12) Patent Application Publication
(10) Pub. No.: US 2013/0052171 A1
(43) Pub. Date: Feb. 28, 2013

(54) IMMUNOMODULATORY ISOLATED LACTOBACILLUS STRAINAND APPLICATION THEREOF

(71) Applicants: Chen-Kai CHANG, TAINAN (TW); Shu-Chen WANG, TAINAN (TW); Pin-Der DUH, TAINAN (TW)

(72) Inventors: Chen-Kai CHANG, TAINAN (TW); Shu-Chen WANG, TAINAN (TW); Pin-Der DUH, TAINAN (TW)

(73) Assignee: CHIA NAN UNIVERSITY OF PHARMACY AND SCIENCE, TAINAN (TW)

(21) Appl. No.: 13/425,441

(22) Filed: Mar. 21, 2012

Related U.S. Application Data
(60) Provisional application No. 61/528,768, filed on Aug. 30, 2011.

(51) Int. Cl. C12N 1/20; A61P 37/00; A61K 35/74

(52) U.S. Cl. 424/93.45; 435/252.9

(57) ABSTRACT

An immunomodulatory isolated Lactobacillus strain is disclosed. The isolated Lactobacillus strain (accession No. CCTCC M 2011279) is purified from plant fermentation products. The isolated Lactobacillus strain, whatever itself or the composition including the same, can specifically enhance the amount of nitric oxide (NO), tumor necrosis factor-α (TNF-α) and interleukin-6 (IL-6), and also specifically enhance phagocytosis of macrophages, thereby enhancing the immunomodulatory ability of a host. Moreover, the isolated Lactobacillus strain of the present invention itself, or a food or its ingredient, a food supplement or a medical composition including the isolated Lactobacillus strain, can enhance the immunomodulatory ability by oral administration.

### RELATED APPLICATIONS

The present application is based on, and claims priority from, U.S. provisional application Ser. No. 61/528,768, filed Aug. 30, 2011, the disclosure of which is hereby incorporated by reference herein in its entirety.

### SEQUENCE LISTING

The sequence listing submitted via EFS, in compliance with 37 CFR §1.52 (e)(5), is incorporated herein by reference. The sequence listing text file submitted via EFS contains the file “TWT02114US-SeqLising.txt”, created on Mar. 19, 2012, which is 1932 bytes in size.

### BACKGROUND

1. Field of Invention

The present invention relates to an isolated Lactobacillus strain and an application thereof. More particularly, the present invention relates to an immunomodulatory isolated Lactobacillus strain and an application thereof.

2. Description of Related Art

Lactic acid bacteria (LAB) are named as such because most of their members convert lactose and other sugars into lactic acid. LABs are also a genus of Gram-positive facultative anaerobic or microaerophilic bacteria, and they are widely applied in fermentation of food industry.

Typically, LABs include main members of the genera Lactobacillus, Leuconostoc, Pediococcus, Lactococcus and Streptococcus, and other members of the genera Aerococcus, Carnobacterium, Enterococcus, Oenococcus, Sporolactobacillus, Teragenococcus, Vagococcu and Weisella. Most LAB strains belong to the genus Lactobacillus. The aforementioned LAB strains belong to the order Lactobacillales, some strains of which can serve as probiotic bacteria. In current studies, two principal kinds of probiotic bacteria, members of the genera Lactobacillus and Bifidobacterium, have been studied in detail.

LABs have been evaluated in recent studies with respect to improvement of allergy-related diseases and gastrointestinal discomfort. However, the raw material for screening LAB is originated mostly from animals (for example, milk, animal body or human body) but less from fermented plant materials (for example, conventionally fermented plant materials).

Therefore, it is necessary to provide an immunomodulatory isolated Lactobacillus strain that is isolated and purified from a fermented plant material, thereby being applied on a food or its ingredient, a food supplement or a medical composition.

### SUMMARY

An immunomodulatory isolated Lactobacillus strain is provided, which is isolated and purified from a fermented plant material.

Moreover, an immunomodulatory composition is provided, which comprises the aforementioned isolated Lactobacillus strain of Lactobacillus plantarum or together with another probiotic microorganism strain. Through oral administration of the isolated Lactobacillus strain, it can specifically enhance the relative amount of nitric oxide (NO), tumor necrosis factor-α (TNF-α) and interleukin-6 (IL-6), and also specifically enhance phagocytosis of a macrophage cell line RAW264.7, thereby enhancing the immunomodulatory ability of a host. Therefore, the isolated Lactobacillus strain can be applied to a food or its ingredient, food supplement or a medical composition.

Accordingly, the invention provides an immunomodulatory isolated Lactobacillus strain is provided. In an embodiment, the isolated Lactobacillus strain is exemplified as Lactobacillus plantarum deposited at the China Center for Type Culture Collection of Wuhan University in China under accession No.: CCTCC M 2011279.

Moreover, the invention further provides an immunomodulatory composition. In an embodiment, the composition comprises an isolated Lactobacillus strain of exemplified as Lactobacillus plantarum (CCTCC M 2011279). An effective dosage of the isolated Lactobacillus strain can specifically enhance the relative amount of nitric oxide (NO), tumor necrosis factor-α (TNF-α) and interleukin-6 (IL-6), and also specifically enhance phagocytosis of a macrophage cell line.

According to an embodiment, the aforementioned isolated Lactobacillus strain is live or inactivate.

According to an embodiment, the aforementioned composition is for use as a food or an ingredient thereof, a food supplement or a medical composition.

According to an embodiment, the aforementioned isolated Lactobacillus strain is further together with another probiotic microorganism strain.

With application to the immunomodulatory isolated Lactobacillus strain of the present invention, which is isolated and purified from the fermented plant material, it can be applied for use in the food or its ingredient, the food supplement or the medical composition, thereby enhancing the immunomodulatory ability of a host.

It is to be understood that both the foregoing general description and the following detailed description are by examples, and are intended to provide further explanation of the invention as claimed.

### DETAILED DESCRIPTION

Reference will now be made in detail to the present embodiments of the invention, examples of which are illustrated in the accompanying drawings. Wherever possible, the same reference numbers are used in the drawings and the description to refer to the same or like parts.

Accordingly, the present invention provides an immunomodulatory isolated Lactobacillus strain is provided, which is isolated and purified from a fermented plant material, and the isolated Lactobacillus strain has immunomodulatory function.

Before proceeding further, it is appropriate to refer the isolated Lactobacillus strain described herein that is Lactobacillus plantarum deposited at the China Center for Type Culture Collection (CCTCC, Wuhan University, Wuhan 430072, People's Republic of China) under the accession number of CCTCC M 2011279 on Aug. 8, 2011.

The artisan in this art is familiar with the isolated Lactobacillus strain CCTCC M 2011279 obtained by the prior methods or the following methods of isolation, screening and culture. Moreover, the isolated Lactobacillus strain can be further determined by species, functions and other related applications.

1. Isolation and Culture of Lactobacillus Strain

In an embodiment, the isolated Lactobacillus strain CCTCC M 2011279 was isolated and purified from a fermented plant material. The term “fermented plant material,” described herein refers to a fermented product that includes the plant material itself, a fermented liquid and other uncertain remains unseparated therefrom or any combination thereof all of which is fermented from a plant material by using lactic acid bacteria. In an example, the plant material may be exemplified as vegetables or fruits. In another example, the resultant fermented plant material may be pickled vegetables or fermented liquid thereof, for example, lactic fermentation products and fermented liquids thereof such as Kimchi, sour cabbage, salted sour cabbage, preserved bamboo shoot or the like.

Next, the plant-fermented liquid was serially diluted, inoculated into a screening medium and incubated at 30° C. under an anaerobic condition for 2 to 3 days. The screening medium was any commercially available calcium salt-containing medium, for example, the MRS agar plate that includes 0.5 wt % of calcium carbonate (CaCO₃) and 1.4 wt % of agar in MRS broth medium (Lactobacillus broth according to de Man, Rogosa and Sharpe; final pH 6.2±0.2). Later, a single isolated colony was picked, streaked out on a sterile plate and incubated (streak plating). The streak plating for isolating single colonies was repeated as many times as necessary until pure cultures of single colonies had been obtained, in which each single colony was referred to an isolated strain. And then, those colonies of the isolated strains were transferred to MRS slant agar respectively, and stored at 4° C. or by lyophilization (optionally added with 15 wt % of non-fat milk as a protecting agent).

2. Species Identification of Lactobacillus Strain

The aforementioned isolated strains were subjected to the first identification step that includes various conventional tests, thereby screening out one or more isolated Lactobacillus strains. In an example, the first identification step includes the conventional tests of Gram staining, microscopy examination, catalase tests, acid/gas production test of glucose fermentation, qualitification test for lactic acid and/or other tests, so as to identify the “genus” of each isolated strain. The Gram staining, microscopy examination, catalase tests, acid/gas production test of glucose fermentation, and qualitification test for lactic acid were well known by the artisan in this art of the present invention rather than being recited in detail herein. In another example, the isolated strain was a Gram-positive, catalase-negative and lactic acid-positive Lactobacillus strain.

And then, the isolated Lactobacillus strain were subjected to the second identification step that involves identification of physiological and biochemical characteristics of Lactobacillus bacteria, in which the isolated Lactobacillus strain was compared with a standard Lactobacillus strain such as Lactobacillus plantarum ATCC 14917 by using commercially available products, for example, API® 50 CHL strip (bioMérieux Inc., France) or other functionally equivalent products, thereby identifying the “species” of the isolated Lactobacillus strain. Alternatively, the second identification step involves identification of genetic characteristics of Lactobacillus bacteria, in which the genomic DNA of the isolated Lactobacillus strain was purified and subjected to 16S rDNA sequence analysis. The sequence of the 16S rDNA of the isolated Lactobacillus strain and the known 16S DNA sequences of the Genbank were compared to determine the sequence similarities therebewteen, thereby identifying the “species” of the isolated Lactobacillus strain.

In an example, the isolated Lactobacillus strain may include but not be limited to Lactobacillus plantarum deposited at the CCTCC under the accession number of CCTCC M 2011279.

3. Functional Identification and Application of Lactobacillus Strain

The isolated Lactobacillus strain isolated and purified from the fermented plant material has immunomodulatory function evidenced by in vitro experiment.

The term “immunomodulatory function” described herein refers to specifically enhance bioactivity of a macrophage, thereby regulating the immunomodulatory function of a host.

In detail, the macrophage is a type of antigen-presenting cells (APCs) and is also important in the nonspecific immune response. The macrophage can phagocytize and kill many pathogens. When the macrophage is activated by interferon-γ (INF-γ), it can be more effectively kill tumor cells or virus-infected cells. In addition, the macrophage can secret many cytokines, for modulating the immune responses and enhancing the proliferation and killing activity of lymphocytes.

The bacterial cells of the immunomodulatory isolated Lactobacillus strain can specifically enhance the amount of nitric oxide (NO), tumor necrosis factor-α (TNF-α) and interleukin-6 (IL-6), and also specifically enhance phagocytosis of macrophages, thereby enhancing the immunomodulatory ability of a host. Moreover, the bacterial cells of the isolated Lactobacillus strain of the present invention themselves, or a food or its ingredient, a food supplement or a medical composition including the bacterial cells of the isolated Lactobacillus strain, can enhance the immunomodulatory ability by oral administration.

It is supplemented that, the isolated Lactobacillus strain of the aforementioned embodiments is live or inactivate while being applied to the production of the compositions, and the isolated Lactobacillus strain has an effective amount of the immunomodulatory activity. Moreover, the isolated Lactobacillus strain of the aforementioned embodiments is also lyophilized and may further include other ingredients, for example, glucose, maltodextrin, baby milk, fructo-oligosaccharides, magnesium stearate, yogurt spices, other uncertain remains unseparated therefrom or any combination thereof, as well as formulation techniques for preparing the oral composition, are known to a person skilled in the art. Preferably the oral composition comprises a dosage of 10⁶ to 10¹¹ colony-forming units (cfu) of the isolated Lactobacillus strain per gram.

In another embodiment, the isolated Lactobacillus strain is further together with at least one another probiotic microorganism strain, so as to enhance the immunomodulatory ability or other functions of a host by oral administration. In an example, the another probiotic microorganism strain may include but be not limited to Lactobacillus acidophilus, Lactobacillus bifidus, Lactobacillus brevis, Lactobacillus bulgaricus, Lactobacillus casei, Lactobacillus causasicus, Lactobacillus gasseti, Lactobacillus johnsonii, Lactobacillus lactis, Lactobacillus rhamnosus, Lactobacillus salivarius or any combination thereof.

Thereinafter, various applications of the isolated Lactobacillus strain will be described in more details referring to several exemplary embodiments below, while not intended to be limiting. Thus, one skilled in the art can easily ascertain the essential characteristics of the present invention can make various changes and modifications of the invention to adapt it to various usages and conditions.

### Example 1

### 1. Isolation of Isolated Lactobacillus Strain

In this EXAMPLE, the isolated Lactobacillus strain was isolated from freshly fermentated juices of fermented plant materials such as various kinds of lactic acid fermentated vegetables. In detail, the fermented juices were serially diluted 10-fold with the water under a sterile environment in this EXAMPLE. Next, 1 mL bacteria suspensions with three serially diluted concentrations (for example, 10⁻⁵, 10⁻⁶ and 10⁻⁷-fold of the original concentration) were selected and added into the screening medium in sterile culture dishes, respectively. The screening medium, for example, the solid medium of MRS broth medium (Lactobacillus MRS broth, HiMedia Labratories PVT, LTD., India; final pH 6.2±0.2) including 0.5 wt % of calcium carbonate (CaCO₃) and 1.4 wt % of agar, had been autoclaved and cooled down to 45° C. to 50° C. After the incubation of 2 days to 3 days at 30° C. approximately, single colonies with clear surrounding zone and various shapes were picked and inoculated on the MRS agar plate by streak plating. The streak plating for isolating single colonies was repeated as many times as necessary until pure cultures of single colonies had been obtained, in which each single colony was referred to an isolated strain. And then, those isolated strains were transferred to MRS slant agar respectively, and stored at 4° C.

### 2. Identification of Isolated Lactobacillus Strain

2.1 The First Identification Step

The pure cultures of the isolated strain were subjected to the first identification step, thereby identifying the “genus” of each isolated strain. In the first identification step, Gram staining, microscopy examination, catalase tests, acid/gas production test of glucose fermentation, qualitification test for lactic acid and/or other tests can be carried out, so as to screen out the Gram-positive, catalase-negative and lactic acid-positive Lactobacillus strain.

The methods of Gram staining and microscopy examination were well known by the artisan in this art of the present invention rather than being recited in detail herein. The artisan in this art is familiar with the methods of the catalase tests, acid/gas production test of glucose fermentation, and qualitification test for lactic acid performed by the prior methods or the following methods.

2.1.1 Catalase Test

The being-identified isolated strain was inoculated to peptone-yeast extract-glucose (PYG) slant agar and incubated at 30° C. for 18 hours to 24 hours approximately. Next, a cultured of a colony was picked by the inoculation loop, put onto a clean slide, added and reacted with 3% to 15% hydrogen peroxide (H₂O₂) on the colony drop by drop. Vigorous bubbling indicated a catalase positive result, and no bubbling indicated a catalase negative result.

2.1.2 Acid/Gas Production Test of Glucose Fermentation

Acid/gas production test of glucose fermentation can be performed by the prior methods or the following methods. The medium for acid/gas production test included peptone-yeast extract (PY) basic medium having 30 g of glucose, 0.5% of Tween-80 and 6.0 g of agar with 1.4 mL of bromocresol purple (1.6 g/100 mL) as a pH indicator, and then the melted medium for acid/gas production test was aliquoted into test tubes. After the suspension of the isolated strain was added into the melted medium drop by drop and mixed well, 2% agar layer was poured on the medium up to 7 mm in thickness approximately and incubated at 30° C. for 6 days approximately. The pH indicator in those media will turn yellow if acid is produced in the fermentation. The bubbles will occur or the 2% agar layer will be moved up if gas is produced in the fermentation.

2.2 The Second Identification Step

2.2.1 API 50 CHL Identification System

The isolated Lactobacillus strains screened by the first identification step were further subjected to the second identification step by using API® 50 CHL identification system. The younger and activated colonies were picked from the isolated Lactobacillus strain screened by the first identification step, inoculated into API® 50 CHL liquid medium (bioMérieux Inc., France), shaked and mixed well. Next, the bacterial suspensions of the isolated Lactobacillus strains and the standard strain (for example, Lactobacillus plantarum ATCC 14917) (90 μL of the bacterial suspension per test well) were added into the test wells of API® 50 CHL strip, respectively, sealed by sterile liquid paraffin (50 μL per test well), and incubated at 30° C. for 48 hours approximately in an anaerobic condition. And then, 80 μL of sterile distilled water was introduced into each honeycomb-like cells in the bottom of the incubation box, for keeping enough moisture in the incubation environment. The standard strain of the EXAMPLE is Lactobacillus plantarum ATCC 14917. Other detail of the operation steps was accorded to the manufacturer's operation manual rather than being recited in detail herein.

Afterward, the “species” of the isolated Lactobacillus strain was identified by visual observation or API Lab Plus software (bioMérieux Inc., France) according to the manufacture's operation manual rather than being recited in detail herein. After the identification of the second identification step, the isolated Lactobacillus strain is identified to Lactobacillus plantarum.

2.2.2 Identification of 16s rDNA Sequence Analysis

2.2.2.1 PCR Primer Pairs

In the EXAMPLE, the 16s rDNA sequence analysis was carried out according to the primer pairs of 16s rDNA gene published by Michael et al. The upstream primer listed as SEQ ID No. 1 in the second raw of TABLE was a bacteria-specific primer 27F. The downstream primer listed as SEQ ID No. 2 in the third raw of TABLE was a universal primer 1492R. The primer pair was synthesized by ScinoPharm Biotech Ltd. and listed as TABLE 1:

TABLE 1

| SEQ ID No. | Sequence |
|---|---|
| 1 | 5′-AGAGTTTGATCMTGGCTCAG-3′ |
| 2 | 5′-GGYTACCTTGTTACGACTT-3′ |

(M = C, A; Y = C, T)

2.2.2.2 16s rDNA Sequence Analysis

The genomic DNA of the isolated Lactobacillus strain was obtained by using a commercially available genomic DNA extraction kit, for example, Tissue and Cell Genomic DNA Purification Kit (Hopegen Biotechnology Development Enterprise, Taichung, Taiwan) according to the manufacture's operation manual, followed by the treatment of proteinase K and ribonuclease (RNase). However, the genomic DNA extraction technology was familiar with the artisan in this art of the present invention rather than being recited in detail herein.

Later, the purified genomic DNA of the Lactobacillus strain was subjected to 16s rDNA polymerase chain reaction (PCR) in which the PCR reaction mixture was exemplified as TABLE 2:

TABLE 2

| | Volume |
|---|---|
| Components | (μL) |
| Genomic DNA | 5 |
| dNTPs (2.5 mM; Bioline, U.S.A.) | 2.5 |
| 10x PCR buffer (Bioline, U.S.A.) | 5 |
| 27F/1492R primer (30 pmole/μl) | 1.6 |
| ExSel high DNA polymerase (5U per reaction; Bioline, U.S.A.) | 0.04 |
| Add sterile double-deionized water to | 50 |

The PCR was carried out in commercially available PCR machine (for example, GeneAmp PCR system 2400, Applied Biosystems, U.S.A.) according to the following conditions. At first, the PCR reaction mixture of TABLE 2 was reacted at 94° C. for 10 minutes approximately, so as to denature double-stranded DNA (dsDNA) template to single-stranded DNA (ssDNA) templates. Next, the PCR reaction mixture was repeated for 30 cycles to 40 cycles, and each cycle included a denaturation step at 94° C. for 1 minute, an annealing step for annealing the primer pair to the ssDNA template at 45° C. for 1 minute, and an extension step at 72° C. for 7 minutes. Alternatively, the PCR reaction mixture was repeated for 35 cycles. After repeating the desired cycles, the PCR reaction mixture was ended with the prolonged last extension step on 72° C. for 1 minute for ensuring the PCR reaction more completely. Later, the PCR reaction product was analyzed by an electrophoresis. The electrophoresis of the PCR product was analyzed by using 1.8% agarose gel that run in a 1× electrophoresis buffer, for example, 1× tris(hydroxymethyl) aminomethane (Tris)/borate/ethylenediaminetetraacetic acid (EDTA) (Tris/borate/EDTA buffer; TBE buffer), for separating the PCR product, confirming its correct size (unshown) and purifying it. The agarose gel electrophoresis, the purification of the PCR product and the like were well known by the artisan in this art of the present invention rather than being recited in detail herein.

The 16S rDNA sequence of the purified PCR product was analyzed by using an automatic DNA sequencer (Applied biosystems 3100/3130XL Genetic Analyzer) in the DNA Sequencing Center of the National Cheng Kung University in Tainan, Taiwan. The resulted nucleotide sequence was listed as SEQ ID No. 3. In comparison with the sequence of the 13^{th} nucleotide to the 987^{th} nucleotide of the 16S rDNA sequence of Lactobacillus plantarum (Genbank accession number AB494717) of the National Center for Biotechnology Information (NCBI) website, the corresponding sequence of the eleventh nucleotide to the 985^{th} nucleotide of the 16S rDNA sequence of the isolated Lactobacillus strain has a sequence similarity of more than 99%.

Through the aforementioned identification, the isolated Lactobacillus strain was identified to Lactobacillus plantarum deposited at the China Center for Type Culture Collection (CCTCC, Wuhan University, Wuhan 430072, People's Republic of China) under the accession number of CCTCC M 2011279.

### Example 2

### Assessment of Immunomodulatory Function of Isolated Lactobacillus Strain

In this EXAMPLE, the immunomodulatory activity of the isolated Lactobacillus strain of EXAMPLE 1 was accessed by using a macrophage cell line RAW264.7.

1. Pre-treatment

1.1 Pre-Treatment of Isolated Lactobacillus Strain

Before proceeding further assessments, the isolated Lactobacillus strain stored at −70° C. was thawed, and the bacterial suspension was inoculated into a freshly prepared MRS medium (MRS broth; de Man, Rogosa, Sharpe medium, Himedia), incubated at 37° C. overnight (18 hours approximately) for activation, 1% by volume of the bacterial suspension was further inoculated to the freshly prepared MRS medium at 37° C. for 18 hours. The twice-activated bacterial cells were used for subsequent assessment.

In this EXAMPLE, the bacterial cells of the isolated Lactobacillus strain in the log-grown phase were centrifuged to remove the supernant. And then, the bacterial cells were rinsed twice by phosphate buffered saline (PBS). After centrifugation, the supernant was removed and the cell pellet was diluted by a freshly complete medium of Dulbecco's modified Eagle's medium (DMEM; invitrogen; U.S.A.) in the absence of antibiotics to the cell density of OD₆₀₀ 1.0 (i.e. 1×10⁹ cells/mL approximately). The bacterial suspension (1×10⁹ cells/mL approximately) was aliquoted to 10 mL centrifugation tube with a screw cap, 1 mL of the bacterial suspension per tube, and the bacterial suspension of every tube was boiled at 100° C. for 1 hours. And then, a part of the bacterial suspension was spreaded on a MRS agar plate and incubated at 37° C. for observing and ensuring no viable bacteria.

1.2 Culture of Macrophages

In this EXAMPLE, a murine macrophage cell line RAW264.7 (ATCC No. TIB-71) was employed to access an immunomodulatory ability of the isolated Lactobacillus strain of EXAMPLE 1. The cryo vial of the macrophage cell line RAW264.7 was taken out from the liquid nitrogen tank and immediately thawed in the water bath tank at 37° C. Next, the cells were transferred from the cryo vial to a 9 cm cell culture petri dish, added with 10 mL complete medium and incubated in the incubator with 5% CO₂ humidified atmosphere at 37° C. When the cells were grown to 80% to 90% of confluence with respect to the area of the cell culture petri dish, the cells were subsultured as follows. After sucking the medium out, 5 mL PBS washed the cells twice, and then the cells were scrapped by using a cell scrapper, so as to suspend cells in 5 mL PBS. When the cell monolayer was scrapped from the bottom of the petri dish, the cell suspension was transferred to 15 mL centrifugation tube. After centrifugation, the supernant was removed and the cell pellet was resuspended by 5 mL of a freshly complete medium of DMEM with 10% FBS. 20 μL of the cell suspension was added with an equivalent volume of trypan blue and viable cells were counted by a hematocytometer. Later, according to requirements of various experiments, the cell suspension was seeded with a desired cell numbers into each well of a microtiter cell plate.

### 2. Assessment of Immunomodulatory Function

Hereinafter, the Lactobacillus casei Shirota was used as a bacteria to control group, and the macrophage cell line RAW264.7 was used as a cell control group.

2.1 Stimulation Experiments of Macrophage

The macrophage cell line RAW264.7 was pre-cultured in 96-well microplate for 24 hours. After removing the medium, the cells were rinsed twice by PBS at 37° C. and added with 100 μL of freshly prepared DMEM (including 10% FBS). Next, 100 μL of dead bacterial cells of the pre-treatment of EXAMPLE 2 (1×10⁹ cells/mL approximately) were added into each well. After the incubation at 37° C. for 24 hours to 72 hours, the supernant was removed and the cells of each well were washed twice by PBS. And then, each well of 96-well microplate was added with 100 μL of methylthiazoletetrazolium (MTT; Sigma-Aldrich, St. Louis, U.S.A.) solution (dissolved in the serum-free DMEM), followed by reacting under darkness in the incubator for 3 hours to 4 hours at 37° C. The supernatant was discarded and 100 μL of dimethyl sulfoxide (DMSO) solution was added and reacted under darkness for 5 minutes to 10 minutes. Afterward, the absorbance at 570 nm (OD₅₇₀ₙₘ) of each well was detected by ELISA reader (VMAX Microplate Reader, Molecular Devices, NJ, U.S.A.), and the absorbance of the dead bacterial cells (Lactobacillus casei Shirota) was referred as 100%. The result was shown in the “cell viability” column of TABLE 3.

2.2 Phagocytosis Assay of Macrophage

When the immune system defects exogenous pathogens, one response is to cause cell inflammation, for attracting the defective cells (for example, macrophages) and cleaning the exogenous pathogens and the damaged cells by the phagocytosis and digestion of the defective cells in the inflammation site. This assay is to access the degree of cell inflammation induced by the isolated Lactobacillus strain (CCTCC M 2011279).

The macrophage cell line RAW264.7 was pre-cultured in 96-well microplate for 24 hours. After removing the medium, the cells were rinsed twice by PBS at 37° C. and added with 100 μL of freshly prepared DMEM (including 10% FBS). Next, 100 μL of dead bacterial cells of the pre-treatment of EXAMPLE 2 (1×10⁹ cells/mL approximately) were added into each well. After the incubation at 37° C. for 3 hours (5% CO₂), the supernant was removed, the cells of each well were added with 100 μL of neutral red-PBS solution (0.075 g/100 mL) and incubated for 1 hour. The cells were rinsed thrice by PBS at 37° C. and added with cell lysis solution (1M acetic acid:ethanol=1:1, v/v), followed by reacting at 37° C. overnight. Afterward, the absorbance at 540 nm (OD₅₄₀ₙₘ) of each well was detected by ELISA reader (VMAX Microplate Reader, Molecular Devices, NJ, U.S.A.), and the absorbance of the dead bacterial cells (Lactobacillus casei Shirota) was referred as 100%. The result was shown in the “phagocytosis activity” column of TABLE 3.

2.3 Assay of Nitric Oxide (NO)

The macrophage cell line RAW264.7 was pre-cultured in 96-well microplate for 24 hours. After removing the medium, the cells were rinsed twice by PBS at 37° C. and added with 100 μL of freshly prepared phenol red-free DMEM (including 10% FBS). Next, 100 μL of dead bacterial cells of the pre-treatment of EXAMPLE 2 (1×10⁹ cells/mL approximately) were added into each well. After the incubation at 37° C. for 24 hours (5% CO₂), 170 μL of the supernant was transferred to another 96-well microplate and added with 170 μL of Griess reagent, followed by reacting under darkness for 10 minutes at 37° C. Afterward, the absorbance at 540 nm (OD₅₄₀ₙₘ) of each well was detected by ELISA reader (VMAX Microplate Reader, Molecular Devices, NJ, U.S.A.), and the absorbance of the dead bacterial cells (Lactobacillus casei Shirota) was referred as 100%. The standard curve was created by various concentrations of NaNO₂ solution (unshown). The result was shown in the “NO relative change” column of TABLE 3.

2.4 Assay of Cytokines (TNF-α, IL-6)

The macrophage cell line RAW264.7 was pre-cultured in 96-well microplate for 24 hours. After removing the medium, the cells were rinsed twice by PBS at 37° C. and added with 100 μL of freshly prepared phenol red-free DMEM (including 10% FBS). Next, 100 μL of dead bacterial cells of the pre-treatment of EXAMPLE 2 (1×10⁹ cells/mL approximately) were added into each well. After the incubation at 37° C. for 24 hours (5% CO₂), the supernant was harvested to detect cytokines (TNF-α, IL-6) therein by using a commercially available detection kit, for example, BD Pharmingen™ ELISA Kit according to the manufacturer's protocols.

(1) Before the assay, each well of the 96-well microplate was coated with 100 μL of anti-mouse monoclonal antibody (capture antibody) and placed at 37° C. in a freezer overnight.

(2) Next day, each well was rinsed thrice by 300 μL of Assay Diluent (including FBS), added with 200 μL of Block Buffer and incubated at room temperature for 1 hour.

(3) Each well was rinsed thrice by 300 μL of Assay Diluent (including FBS), added with 100 μL of test sample and incubated at room temperature for 2 hours.

(4) Each well was rinsed five times by 300 μL of Assay Diluent (including FBS), added with 100 μL of biotinylated anti-mouse TNF-α (detection antibody) plus enzyme-reagent and incubated at room temperature for 1 hour.

(5) Each well was rinsed seven times by 300 μL of Assay Diluent (including FBS), added with 100 μL of TMB (3,3′,5,5″-tetramethylbenzidine) and incubated in the darkness for 30 minutes.

(6) The reaction was terminated by addition of Stop Solution. Afterward, the absorbance at 540 nm (OD₅₄₀ₙₘ) of each well was detected by ELISA reader (VMAX Microplate Reader, Molecular Devices, NJ, U.S.A.).

The absorbance of the dead bacterial cells (Lactobacillus casei Shirota) was referred as 100%. The result was shown in the two columns of “TNF-α relative change” and “IL-6 relative change” of TABLE 3.

All data of the aforementioned EXAMPLES was analyzed by a commercially statistical software (for example, SPSS 12.0). Each data of each group was calculated by at least three samples. The data having P<0.05 was defined as statistical significance. The symbol “*” was referred to the statically significant difference in the isolated Lactobacillus strain as compared with Lactobacillus casei Shirota (P<0.05).

TABLE 3

| | Assays | | | | |
|---|---|---|---|---|---|
| | | | TNF-α | | |
| | Phagocytosis | NO relative | relative | IL-6 relative | |
| | activity | change | change | change | Cell viability |
| Strain | (fold control) | (fold control) | (fold control) | (fold control) | (% of control) |
| L. casei Shirota | 2.75 ± 0.09 | 1.69 ± 0.08 | 1.21 ± 0.13 | 2.92 ± 0.26 | 100.33 ± 9.40 |
| LPS | 1.62 ± 0.12* | 17.02 ± 0.83* | 1.91 ± 0.18 | 5.27 ± 0.09* | 58.60 ± 7.49* |
| Isolated Lactibacillus | 2.79 ± 0.05 | 3.48 ± 1.08* | 5.60 ± 0.41* | 4.37 ± 0.55* | 112.00 ± 34.08 |
| strain (CCTCC M | | | | | |
| 2011279) | | | | | |

As the results shown in the two columns of “Phagocytosis activity” and “Cell viability” of TABLE 3, in comparison with LPS (lipopolysaccharide, one of the cell wall components of Gram's negative bacteria) that caused the cell inflammation, the dead bacterial cells of the pre-treatment of EXAMPLE 2 could enhance the phagocytosis activity rather than causing cell death.

Moreover, in comparison with Lactobacillus casei Shirota, the dead bacterial cells of the pre-treatment of EXAMPLE 2 could specifically increase the relative changes of NO, TNF-α, IL-6 of the macrophage cell line RAW264.7, as shown in the three columns of “NO relative change”, “TNF-α relative change” and “IL-6 relative change” of TABLE 3.

Accordingly, the isolated Lactobacillus strain (CCTCC M 2011279) has immunomodulatory function, as evidenced by all results of the aforementioned EXAMPLES.

In summary, the isolated Lactobacillus strain is isolated and purified from plant fermentation products. The isolated Lactobacillus strain has immunomodulatory function, as evidenced by the results of in vitro experiments. Therefore, the isolated Lactobacillus strain of the present invention can be applied on a food or its ingredient, a food supplement or a medical composition including the isolated Lactobacillus strain. However, it is necessarily supplemented that, specific strains, specific analyzing methods, specific experiments, specific reaction conditions or specific instruments are employed as exemplary embodiments in the present invention, for evaluating the isolated Lactobacillus strain of the present invention that can specific enhance the immunomodulatory function. However, as is understood by a person skilled in the art, the isolated Lactobacillus strain of the present invention can include other another probiotic microorganism strains, other analyzing methods, other tests, other reaction conditions, other equivalent materials or other instruments rather than limiting to the aforementioned examples.

Moreover, the another probiotic microorganism strain may include but be not limited to Lactobacillus acidophilus, Lactobacillus bifidus, Lactobacillus brevis, Lactobacillus bulgaricus, Lactobacillus casei, Lactobacillus causasicus, Lactobacillus gasseti, Lactobacillus johnsonii, Lactobacillus lactis, Lactobacillus rhamnosus, Lactobacillus salivarius or any combination thereof.

In addition, when the isolated Lactobacillus strain of the present invention is applied to production of the medical composition, the food supplement, the food or the ingredient thereof, the isolated Lactobacillus strain may be a live, inactivate or lyophilized form. Furthermore, the isolated Lactobacillus strain of the present invention may include other ingredients, for example, glucose, maltodextrin, baby milk, fructo-oligosaccharides, magnesium stearate, yogurt spices, other uncertain remains unseparated therefrom or any combination thereof, as well as formulation techniques for preparing the oral composition, are known to a person skilled in the art.

According to the embodiments of the present invention, the immunomodulatory isolated Lactobacillus strain, which is advantageously isolated and purified from a fermented plant material, itself or a composition including thereof, can specifically increase the relative changes of NO, TNF-α, IL-6 and the phagocytosis activity of the macrophage cell line RAW264.7 by oral administration, thereby enhancing the immunomodulatory ability of a host and being applied on a food or its ingredient, a food supplement or a medical composition.

As is understood by a person skilled in the art, the foregoing preferred embodiments of the present invention are illustrated of the present invention rather than limiting of the present invention. In view of the foregoing, it is intended to cover various modifications and similar arrangements included within the spirit and scope of the appended claims. Therefore, the scope of which should be accorded the broadest interpretation so as to encompass all such modifications and similar structure.

What is claimed is:

1. An immunomodulatory isolated Lactobacillus strain of Lactobacillus plantarum deposited at the China Center for Type Culture Collection of Wuhan University in China under accession No.: CCTCC M 2011279.

2. An immunomodulatory composition which comprises an isolated Lactobacillus strain of Lactobacillus plantarum (deposited at the China Center for Type Culture Collection of Wuhan University in China under accession No.: CCTCC M 2011279) for specifically enhancing nitric oxide (NO), tumor necrosis factor-α (TNF-α), interleukin-6 (IL-6) and phagocytosis of macrophages.

3. The immunomodulatory composition of claim 2, wherein the composition is for use as a food or an ingredient thereof, a food supplement or a medical composition.

4. The immunomodulatory composition of claim 2, wherein the isolated Lactobacillus strain is live or inactivate.

5. The immunomodulatory composition of claim 2, wherein the isolated Lactobacillus strain is further together with at least one another probiotic microorganism strain.

6. The positive photosensitive resin composition of claim 5, wherein the another probiotic microorganism strain is selected from the group consisting of Lactobacillus acidophilus, Lactobacillus bifidus, Lactobacillus brevis, Lactobacillus bulgaricus, Lactobacillus casei, Lactobacillus causasicus, Lactobacillus gasseti, Lactobacillus johnsonii, Lactobacillus lactis, Lactobacillus rhamnosus, Lactobacillus salivarius and any combination thereof.
